(19) United States
(12) Patent Application Publication
(10) Pub. No.: US 2020/0282014 A1
(43) Pub. Date: Sep. 10, 2020

(54) PERCUTANEOUS ANTI-MICROBIOTA FORMULATIONS

(71) Applicant: Hoffman Technologies LLC, Cranford, NJ (US)

(72) Inventors: Steven Hoffman, Mahway, NJ (US); John Rothman, Lebanon, NJ (US)

(21) Appl. No.: 16/293,165

(22) Filed: Mar. 5, 2019

(51) Int. Cl. A61K 38/14; A61K 47/22; A61K 47/34; A61K 47/10; A61K 47/12; A61K 9/00; A61K 33/18; A61K 31/43; A61P 31/04; A61P 31/12; A61P 31/02

(52) U.S. Cl. CPC A61K 38/14; A61K 47/22; A61K 47/34; A61K 47/10; A61K 47/12; A61P 31/02; A61K 33/18; A61K 31/43; A61P 31/04; A61P 31/12; A61K 9/0014

(57) ABSTRACT

The present disclosure is directed to percutaneous compositions containing an antiseptic, an antibiotic, an antifungal agent, an antihelminth agent, an antiviral agent, or an NSAID.

### TECHNICAL FIELD

The present disclosure is directed to percutaneous compositions containing an antiseptic, an antibiotic, an antifungal agent, an antihelminth agent, an antiviral agent, or an NSAID.

### BACKGROUND

The skin, being the outermost protective barrier of the body, is susceptible to infection by a variety of microbiota such as bacteria, viruses, fungi, amoeba, and protozoans. Treatment of such infections depends critically on the ability to delivery therapeutic agents to the surface and interior of the skin.

For example, elemental iodine (I₂) is an excellent topical antiseptic that kills bacteria, viruses, fungi, amoeba, protozoans and other microbiota to which it is exposed. I₂ is a strong oxidant that was made therapeutically practicable by the discovery of iodophores. An iodophore is iodine in combination with a surfactant, which facilitates the penetration of iodine into susceptible tissues before it is converted by the body into iodide (I⁻), a non-microbicidal form. Iodophores reduce the immediate irritation that I₂ can cause to skin while facilitating its antiseptic properties.

Currently, the preferred iodophore is polyvinylpyrrolidone (PVP) in combination with iodine (PVP-I). This formulation enables a small amount of I₂ to be released in equilibrium with the I₂ that remains associated with the iodophore.

A need exists for formulations that are capable of topical or intradermal delivery of therapeutic agents such as antiseptics, antibiotics, antifungals, antihelminth agents, antiviral agents.

### SUMMARY

The present disclosure is directed to compositions comprising a first component, a second component, a C₁₋₁₀alkyl alcohol, an organic acid having 1 to 25 carbon atoms, and a therapeutic agent that is an antiseptic, an antibiotic, an antifungal agent, an antihelminth agent, or an antiviral agent, or an NSAID, wherein the first and second components are further defined herein. Methods of making and using these compositions are also described.

### DETAILED DESCRIPTION OF ILLUSTRATIVE EMBODIMENTS

The present disclosure may be understood more readily by reference to the following detailed description of desired embodiments and the examples included therein. In the following specification and the claims that follow, reference will be made to several terms which have the following meanings.

As used in the specification and in the claims, the term “comprising” may include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 1 to 10” is inclusive of the endpoints, 1 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

As used herein, “alkyl” refers to straight chain and branched chains having the indicated number of carbon atoms, usually from 1 to 20 carbon atoms, for example 1 to 8 carbon atoms, such as 1 to 6 or 1 to 7 carbon atoms. For example, C₁₋₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “butyl” is meant to include n-butyl, sec-butyl, isobutyl and t-butyl; “propyl” includes n-propyl and isopropyl. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like.

As used herein, “alkenyl” refers to an unsaturated branched or straight-chain alkyl group having at least one carbon-carbon double bond. The group may be in either the cis or trans configuration about the double bond(s). The group may also be an aromatic group, for example, a phenyl or phenylene moiety. Typical alkenyl groups include, but are not limited to, ethenyl; propenyls such as prop-1-en-1-yl, prop-1-en-2-yl, prop-2-en-1-yl (allyl), prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl; phenylene, and the like. In certain embodiments, an alkenyl group has from 2 to 20 carbon atoms.

As used herein, “alkynyl” refers to an unsaturated branched or straight-chain alkyl group having at least one carbon-carbon triple bond derived by the removal of two molecules of hydrogen from adjacent carbon atoms of the parent alkyl. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group has from 2 to 20 carbon atoms.

The present disclosure is directed to compositions that enhance the intradermal and/or transdermal permeation of therapeutic agents such as iodine into the skin. As used herein, the term “transdermal permeation” includes intradermal delivery, percutaneous delivery, and transmucosal delivery, that is, passage through skin or mucosal tissue and into the bloodstream. As used herein in reference to transdermal permeation, the term “enhancing” refers to increasing the rate at which a therapeutic agent traverses the stratum corneum outer layer of the skin or mucosal tissue. These compositions include a first component, a second component, an alcohol, an organic acid, and, optionally, water. The compositions of the disclosure further comprise a therapeutic agent.

According to the disclosure, the first component comprises one of the following;
a compound of formula I

R—(OCH₂CH₂)_{y}—OH (I)
wherein R is C₁₋₂₀alkyl, C₂₋₂₀alkenyl; or C₂₋₂₀alkynyl; and y is 1 to 25;
a tetrafunctional block copolymer surfactant terminating in primary hydroxyl groups;
a sorbitan derivative;
a C₈₋₁₀alkyl ammonium salt;
a compound of formula II

HO—(CH₂CH₂O)ₘ—C(CH₃)(C₄H₉)—C≡C—C(CH₃)(C₄H₉)—(OCH₂CH₂)ₙ—OH (II)
wherein m and n are each independently 1 to 25;
or a combination thereof.

In preferred embodiments of the disclosure, the first component is a compound of formula I. In some embodiments, R is C₁₋₂₀alkyl, which can either be a straight chain or branched alkyl. Preferred compounds of formula I wherein R is C₁₋₂₀alkyl include, for example, is cetomacrogol 1000; octadecan-1-ol, ethoxylated; polyoxyethylene(12)tridecyl ether; polyoxyethylene(10)tridecyl ether; fatty alcohol polyoxyethylene ether, polyoxyethylene branched nonylcyclohexyl ether (TRITON N-101), nonaethylene glycol monododecyl ether, 23-{[4-(2,4,4-trimethyl-2-pentanyl)cyclohexyl]oxy}-3,6,9,12,15,18,21-heptaoxatricosan-1-ol, and combinations thereof. Nonaethylene glycol monododecyl ether is particularly preferred.

In other embodiments, R is C₂₋₂₀alkenyl, which can either be a straight chain or branched alkenyl. Preferred compounds of formula I wherein R is C₂₋₂₀alkenyl include, for example, polyoxyl(10)oleyl ether, polyethylene glycol tert-octylphenyl ether (TRITON X-100), and combinations thereof

In yet other embodiment, R is C₂₋₂₀alkynyl, which can either be a straight chain or branch alkynyl.

In those embodiments wherein the first component is a compound of formula I, y is 1 to 25. In preferred embodiments, y is 5 to 15, preferably 8 to 10, with 9 being particularly preferred. In other embodiments, y is 1, 2, 3, 4, 5, 6, 7, 8, 9, 10, 11, 12, 13, 14, 15, 16, 17, 18, 19, 20, 21, 22, 23, 24, or 25.

In other aspects of the disclosure, the first component is a tetrafunctional block copolymer surfactant terminating in primary hydroxyl groups. Such compounds are commercially available under the tradename TETRONIC and include ethylenediaminetetrakis(ethoxylate-Block-propoxylate).

In other embodiments of the disclosure, the first component is a sorbitan derivative, for example, polyoxyethylene sorbitan tetraoleate, 1,4-anhydro-6-O-palmitoyl-D-glucitol (sorbitan, monohexadecanoate), a polyethylene glycol sorbitan monolaurate (e.g., TWEEN 20, TWEEN 40, TWEEN 60, TWEEN 85), and combinations thereof.

In still other embodiments of the disclosure, the first component is a C₈₋₁₀alkyl ammonium salt, for example, methyltrialkyl(C₈-C₁₀)ammonium chloride (ADOGEN 464).

In other embodiments, the first component is a compound of formula II.

The compositions of the disclosure can comprise from about 0.1 vol. % to about 40 vol. % of the first component. In preferred embodiments, the compositions comprise from about 1 vol. % to about 40 vol. % of the first component. In other embodiments, the compositions comprise from about 0.1 vol. % to about 5 vol. % of the first component. For example, the compositions can comprise about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or about 40 vol. % of the first component.

According to the disclosure, the compositions also include a second component that comprises one of the following:
a compound of the formula III

R²—N(R¹)—C(O)—R³ (III)
wherein
each R¹ is independently H or C₁₋₃alkyl; and
R² and R³ are independently C₁₋₇alkyl or together with the atoms to which they are attached, form a lactam having 3 to 10 carbon atoms,
a sulfoxide;
a urea;
ethyl acetate;

or a combination thereof.

In preferred embodiments, the second component is compound of formula III. In some embodiments, R¹ is H. In other embodiments, R¹ is methyl, ethyl, propyl, or isopropyl, with methyl being particularly preferred.

In those embodiments wherein R² and R³ are independently C₁₋₇alkyl, each of R² and R³ is independently methyl, ethyl, propyl, isopropyl, butyl, s-butyl, t-butyl, pentyl, hexyl, or heptyl.

Preferably, R² and R³, together with the atoms to which they are attached, form a lactam having 3 to 10 carbon atoms. For example, the lactam can include 3, 4, 5, 6, 7, 8, 9, or 10 carbons, which can be a part of the lactam ring or which can form exocyclic branching. Examples of preferred lactams include pyrrolidones such as 2-pyrrolidone, 1-methyl-2-pyrrolidone (NMP), 5-methyl-2-pyrrolidone, and 1-ethyl-2-pyrrolidone. Preferably, the lactam is 1-methyl-2-pyrrolidinone (i.e., NMP) or 2-pyrrolidone.

Embodiments of the compositions of the invention which contain both iodine and a pyrrolidone may contain a non-covalent complex between the iodine and the pyrrolidone. These non-covalent complexes are referred to as iodophores. In some embodiments, the pyrrolidone is NMP. In some embodiments, iodophore comprises NMP and iodine, i.e., is an I₂-NMP iodophore. The presence of an iodophore can be established by spectroscopic means, such as UV/Vis spectroscopy, using techniques known by those skilled in the art.

In some embodiments, the compositions of the invention contain both an I₂-NMP iodophore and a polyvinylpyrrolidone (PVP)-iodine (PVP-I) iodophore.

In some embodiments, the second component is a sulfoxide, for example, dimethyl sulfoxide.

In other embodiments, the second component is a urea, for example an imidazolidinone.

The compositions of the disclosure can comprise from about 0.01 vol. % to about 10 vol. % of the second component. In preferred embodiments, the compositions comprise from about 0.01 vol. % to about 5 vol. % of the second component. In other embodiments, the compositions comprise from about 0.01 vol. % to about 4 vol. % of the second component. For example, the compositions can comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or about 10 vol. % of the second component.

In some embodiments of the disclosure, the ratio, by volume, of the first component to the second component is about 10:1 to about 4:1.

Alcohols for use in the compositions of the disclosure include C₁₋₁₀alkyl alcohols having at least one —OH moiety or at least two —OH moieties. For example, preferred alcohols include glycerol, propylene glycol, methanol, ethanol, isopropanol, 1-propanol, butanol, t-butanol, pentanol, 1-octanol, and combinations thereof, with ethanol being particularly preferred.

The compositions of the disclosure can comprise from about 0.1 vol. % to about 99 vol. % of the C₁₋₁₀ alkyl alcohol. In some preferred embodiments, the compositions comprise from about 1 vol. % to about 50 vol. % of the C₁₋₁₀ alkyl alcohol. In other embodiments, the compositions comprise from about 0.1 vol. % to about 5 vol. % of the C₁₋₁₀ alkyl alcohol. In other preferred embodiments, the compositions comprise about 90 to about 99 vol. % of the C₁₋₁₀ alkyl alcohol. For example, the compositions can comprise about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 95, 98, or about 99 vol. % of the C₁₋₁₀ alkyl alcohol.

The compositions of the disclosure also include an organic acid having 1 to 25 carbon atoms. For example, organic acids for use in the disclose compositions include acetic acid, ascorbic acid, lactic acid, glycolic acid, propionic acid, and combinations thereof.

Other organic acids for use in the disclosure include fatty acids. As used herein, the term “fatty acid” has its ordinary meaning as would be understood by a person of ordinary skill in the art and includes a molecule having a carboxylic group and a hydrocarbon chain. Descriptions of the number of carbon atoms in a fatty acid herein refer to the number of carbon atoms in the hydrocarbon chain of the fatty acid, irrespective of whether the hydrocarbon chain is straight or branched.

As used herein, the term “fatty acid” includes saturated fatty acids, which do not contain any double or triple bonds in the hydrocarbon chain. Saturated fatty acids include, but are not limited to propionic acid (C3) (by way of example, C3 indicates propionic acid has 3 carbon atoms in its hydrocarbon chain; the number of carbon atoms in the hydrocarbon chain of other example fatty acids is denoted in analogous fashion herein), butyric acid (C4), valeric acid (C5), caproic acid (C6), enanthic acid (C7), caprylic acid (C8), pelargonic acid (C9), capric acid (C10), undecylic acid (C11), lauric acid (C12), tridecylic acid (C13), myristic acid (C14), pentadecylic acid (C15), palmitic acid (C16), margaric acid (C17), stearic acid (C18), isostearic acid (C18), nonadecylic acid (C19), arachidic acid (C20), heneicosylic acid (C21), behenic acid (C22), tricosylic acid (C23), lignoceric acid (C24), pentacosylic acid (C25), cerotic acid (C26), heptacosylic acid (C27), montanic acid (C28), nonacocylic acid (C29), melissic acid (C30), henatriacontylic acid (C31), lacceroic acid (C32), psyllic acid (C33), geddic acid (C34), ceroplastic acid (C35) and hexatriacontylic acid (C36).

As used herein, the term “fatty acid” also includes monounsaturated fatty acids, which contain one double or triple bond in the hydrocarbon chain, and polyunsaturated fatty acids, which contain more than one double and/or triple bond in the hydrocarbon chain. Such acids include, but are not limited to the omega 3, omega 6, omega 9 fatty acids, other fatty acids such as myristoleic and palmitoleic acid and conjugated fatty acids. Examples of monounsaturated and polyunsaturated fatty acids include but are not limited to, (a) omega 3 fatty acids, such as hexadecatrienoic acid (C16:3); (by way of example, C16:3 indicates hexadecatrienoic acid has 16 carbon atoms in its hydrocarbon chain and 3 double bonds; the number of carbon atoms and double bonds in the hydrocarbon chain of other example unsaturated fatty acids is denoted in analogous fashion herein), alpha linolenic acid (C18:3) and eicosapentanoic acid (20:5), (b) omega 6 fatty acids, such as linoleic acid (18:2), docosadienoic acid (C22:2), arachidonic acid (C20:4) and tetracosatetraenoic acid (C24:5), (c) omega 9 fatty acids, such as oleic acid (C18:1), eicosenoic acid (C20:1) and nevronic acid (C24:1), and (d) conjugated fatty acids such as rumenic acid (C18:2), eleostatic acid (C18:3), and rumelenic acid (C18:3).

As used herein, the term “fatty acid” also includes branched fatty acids. Examples of branched fatty acids include, but are not limited to, monomethyl branched fatty acids, such as 14-methyl pentadecanoic acid, 6-methyl caprylic acid, 4-methyl-3-pentenoic acid, (pyroterebic acid), 2-methyl-2E-butenoic acid (tiglic acid), 2-methyl-2Z-butenoic acid (angelic acid), multimethyl branched acids, isoprenoid fatty acids (vittatalactone, all-trans-retinoic acid), branched methoxy fatty acids and hydroxy and other fatty acids such as 2-hydroxyoctanoic acid and 4-oxopentanoic acid.

The compositions of the disclosure can comprise from about 0.01 vol. % to about 15 vol. % of the organic acid. In some embodiment, the compositions comprise from about 1 vol % to about 15 vol % of the organic acid. In preferred embodiments, the compositions comprise from about 0.01 vol. % to about 5 vol. % of the organic acid. In other embodiments, the compositions comprise from about 0.01 vol. % to about 3 vol. % of the organic acid. For example, the compositions can comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or about 15 vol. % of the organic acid.

Compositions of the disclosure can be anhydrous. As used herein, “anhydrous” refers to compositions comprising less than 1 vol. % of water, preferably less than 0.05 vol. % or less than 0.025 vol. % of water. Methods of determining water content are known in the art.

Compositions of the disclosure can include water. In some embodiments, the compositions can comprise up to 99 vol. % of water. In still other aspects, the compositions can comprise 5, 10, 20, 30, 40, 50, 60, 70, 80, 90, 95, 99, or 99.2 vol. % of water. In other embodiments, the compositions can comprise 90, 91, 92, 93, 94, 95, 96, 97, 98, 99, or 99.2 vol. % of water.

Compositions of the disclosure that include water can optionally contain one or more physiologically acceptable salts. While not being bound by any particular theory, it is believed that controlling the amount of salt that is present allows one to control the depth to which the present composition penetrate skin, with the concentration of salt having a generally inverse relationship to the penetration depth. Salts for use in the compositions include, but are not limited to, sodium chloride, potassium chloride, potassium iodide, sodium iodide, and mixtures thereof. A preferred form of sodium chloride is bacteriostatic sodium chloride solution.

In some embodiments, the compositions of the present invention comprise about 32-36 vol. % of the first component; about 2-4 vol. % of the second component; about 40-48 vol. % of the C₁₋₁₀alkyl alcohol; about 6-12 vol. % of the organic acid; and a therapeutic agent.

In other embodiments, the composition comprises about 3.2-3.6 vol. % of the first component; about 0.2-0.4 vol. % of the second component; about 4.0-92 vol. % of the C₁₋₁₀alkyl alcohol; about 0.6-1.2 vol. % of the organic acid; and a therapeutic agent.

In other embodiments, the composition comprises about 3.2-3.6 vol. % of the first component; about 0.2-0.4 vol. % of the second component; about 4.0-4.8 vol. % of the C₁₋₁₀alkyl alcohol; about 0.6-1.2 vol. % of the organic acid; about 80-92 vol. % water, and a therapeutic agent.

In other embodiments, the composition comprises about 0.32-0.36 vol. % of the first component; about 0.02-0.04 vol. % of the second component; about 0.40-99.2 vol. % of the C₁₋₁₀alkyl alcohol; about 0.06-0.12 vol. % of the organic acid; and a therapeutic agent.

In other embodiments, the composition comprises about 0.32-0.36 vol. % of the first component; about 0.02-0.04 vol. % of the second component; about 0.40-0.48 vol. % of the C₁₋₁₀alkyl alcohol; about 0.06-0.12 vol. % of the organic acid; about 80-92 vol. % water, and a therapeutic agent.

In other embodiments, the compositions of the present invention comprise about 32 vol. % of nonaethylene glycol monododecyl ether; about 3 vol. % of 1-methyl-2-pyrrolidone; about 43 vol. % of ethanol; about 7 vol. % of linoleic acid; and a therapeutic agent.

In other embodiments, the compositions of the present invention comprise about 3.2 vol. % of nonaethylene glycol monododecylether; about 0.3 vol. % of 1-methyl-2-pyrrolidone; about 94.3 vol. % of ethanol; about 0.7 vol. % of linoleic acid; and about 1.5 vol. % of a therapeutic agent.

In other embodiments, the compositions of the present invention comprise about 3.2 vol. % of nonaethylene glycol monododecyl ether; about 0.3 vol. % of 1-methyl-2-pyrrolidone; about 88.8 vol. % of ethanol; about 0.7 vol. % of linoleic acid; and about 7 vol. % of a therapeutic agent.

In other embodiments, the compositions of the present invention comprise about 3.2 vol. % of nonaethylene glycol monododecyl ether; about 0.3 vol. % of 1-methyl-2-pyrrolidone; about 4.3 vol. % of ethanol; about 0.7 vol. % of linoleic acid; about 90 vol. % water, and about 1.5 vol. % of a therapeutic agent.

In other embodiments, the compositions of the present invention comprise about 3.2 vol. % of nonaethylene glycol monododecyl ether; about 0.3 vol. % of 1-methyl-2-pyrrolidone; about 4.3 vol. % of ethanol; about 0.7 vol. % of linoleic acid; about 84.5 vol. % water and about 7 vol. % of a therapeutic agent.

In other embodiments, the compositions of the present invention comprise about 0.32 vol. % of nonaethylene glycol monododecyl ether; about 0.03 vol. % of 1-methyl-2-pyrrolidone; about 98.1 vol. % of ethanol; about 0.07 vol. % of linoleic acid; and about 1.5 vol. % of a therapeutic agent.

In other embodiments, the compositions of the present invention comprise about 0.32 vol. % of nonaethylene glycol monododecyl ether; about 0.03 vol. % of 1-methyl-2-pyrrolidone; about 92.6 vol. % of ethanol; about 0.07 vol. % of linoleic acid; and about 7 vol. % of a therapeutic agent.

In other embodiments, the compositions of the present invention comprise about 0.32 vol. % of nonaethylene glycol monododecyl ether; about 0.03 vol. % of 1-methyl-2-pyrrolidone; about 0.43 vol. % of ethanol; about 0.07 vol. % of linoleic acid; about 97.7 vol. % water, and about 1.5 vol. % of a therapeutic agent.

In other embodiments, the compositions of the present invention comprise about 0.32 vol. % of nonaethylene glycol monododecyl ether; about 0.03 vol. % of 1-methyl-2-pyrrolidone; about 0.43 vol. % of ethanol; about 0.07 vol. % of linoleic acid; about 92.2 vol. % water, and about 7 vol. % of a therapeutic agent. The compositions of the disclosure include a therapeutic agent. As used herein, the term “therapeutic agent” refers to an antiseptic, an antibiotic, an antifungal agent, an antihelminth agent, or an antiviral agent, or an NSAID, that upon administration to a patient in a therapeutically effective amount, provides a therapeutic benefit to the patient. Those skilled in the art will appreciate that the term “therapeutic agent” is not limited to materials that have received regulatory approval.

The compositions of the disclosure in which the therapeutic agent is an antiseptic, an antibiotic, an antifungal agent, an antihelminth agent, or an antiviral agent contain the therapeutic agent in an amount that is suitable for killing microbiota. In some embodiments, the compositions of the disclosure comprise from about 0.01 vol % to about 10 vol % of the therapeutic agent. For example, the compositions can comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 vol % of the therapeutic agent.

The compositions of the disclosure in which the therapeutic agent is an NSAID contain the therapeutic agent in an amount that is suitable for reducing inflammation or pain. In some embodiments, the compositions of the disclosure comprise from about 0.01 vol % to about 10 vol % of the NSAID. For example, the compositions can comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 vol % of the NSAID.

The therapeutic agent in the compositions of the disclosure is an antiseptic, an antibiotic, an antifungal agent, an antihelminth agent, or an antiviral agent, or an NSAID.

In some embodiments, the therapeutic agent is an antiseptic, for example, iodine, chlorhexidine gluconate, octenidine dihydrochloride, polyhexamethylene biguanide, or boric acid, or pharmaceutically acceptable salts thereof. In some embodiments, the antiseptic is iodine.

In some embodiments of the compositions of the disclosure that comprise iodine (I₂), the iodine is present in from about 0.01 vol % to about 7 vol % of iodine. For example, the compositions can comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, vol % iodine.

In some embodiments, the therapeutic agent is an antibiotic, for example, amikacin, aminoglycosides, amoxicillin, amoxicillin/clavulanate, ampicillin, ampicillin/sulbactam, arsphenamine, azithromycin, azlocillin, aztreonam, bacitracin, bactrium, capreomycin, carbapenems, cefaclor, cefadroxil, cefalexin, cefamandole, cefazolin, cefdinir, cefditoren, cefepime, cefepime, cefixime, cefmetazole, cefonicid, cefoperazone, cefotaxime, cefotetan, cefoxitin, cefpodoxime, cefprozil, ceftaroline, ceftaroline fosamil, ceftazidime, ceftazidime, ceftibuten, ceftizoxime, ceftobiprole, ceftobiprole, ceftobiprole, ceftolozane/tazobactam, ceftriaxone, cefuroxime, cephalothin, cephapirin, cephradine, chloramphenicol, ciprofloxacin, clarithromycin, clindamycin, clindamycin, clofazimine, colistin, cycloserine, dalbavancin, dalbavancin, dapsone, daptomycin, daptomycin, daptomycin, demeclocycline, dicloxacillin, doripenem, doxycycline, doxycyline, enoxacin, ertapenem, erythromycin, ethambutol, ethionamide, fidaxomicin, flucloxacillin, fluoroquinolones, fosfomycin, furazolidone, fusidic acid, fusidic acid, gatifloxacin, geldanamycin, gemifloxacin, gentamicin, grepafloxacin, herbimycin, imipenem/cilastatin, isoniazid, kanamycin, levofloxacin, lincomycin, linezolid, linezolid, linezolid, lomefloxacin, loracarbef, loracarbef, mafenide, meropenem, metacycline, methicillin, metronidazole, mezlocillin, minocycline, moxalactam, moxifloxacin, mupirocin, mupirocin, nadifloxacin, nafcillin, nalidixic acid, neomycin, netilmicin, nitrofurantoin, norfloxacin, ofloxacin, oritavancin, oritavancin, oxacillin, oxytetracycline, paromomycin, penicillin g, penicillin g, penicillin v, piperacillin, piperacillin/tazobactam, piperacillin/tazobactam, platensimycin, polymyxin b, posizolid, pyrazinamide, quinupristin/dalfopristin, radezolid, rifabutin, rifampicin, rifapentine, rifaximin, roxithromycin, silver sulfadiazine, sparfloxacin, spectinomycin, spiramycin, streptogramins, streptomycin, streptomycin, sulfacetamide, sulfadiazine, sulfadimethoxine, sulfamethizole, sulfamethoxazole, sulfanilimide, sulfasalazine, sulfisoxazole, sulfonamidochrysoidine, tedizolid, teicoplanin, telavancin, telavancin, telithromycin, temafloxacin, temocillin, tetracycline, thiamphenicol, ticarcillin, ticarcillin/clavulanate, ticarcillin/clavulanic acid, tigecycline, tigecycline, tigecycline, tinidazole, tobramycin, torezolid, trimethoprim, trimethoprim-sulfamethoxazole (co-trimoxazole) (tmp-smx), trovafloxacin, vancomycin, or vancomycin, or pharmaceutically acceptable salts thereof.

In some embodiments, the antibiotic is vancomycin, bactrium, doxycyline, ceftobiprole, ceftaroline, clindamycin, dalbavancin, daptomycin, fusidic acid, linezolid, mupirocin, oritavancin, tedizolid, telavancin, or tigecycline, or pharmaceutically acceptable salts thereof.

In some embodiments, the compositions of the invention comprise two or more different classes of therapeutic agent. In some embodiments, the compositions of the invention contain an antiseptic and an antibiotic. In some embodiments, the antiseptic is iodine and the antibiotic is vancomycin. In other embodiments, the antiseptic is iodine and the antibiotic is amoxicillin.

In some embodiments, the therapeutic agent is an antifungal, for example, abafungin, acrisorcin, albaconazole, amorolfin, butenafine, naftifine, terbinafine, amphotericin b, anidulafungin, aurones, benzoic acid, bifonazole, butoconazole, candicidin, caspofungin, castellani's paint, ciclopirox, clioquinol, clotrimazole, coal tar, copper(ii) sulfate^{II}, crystal violet, econazole, efinaconazole, epoxiconazole, fenticonazole (base, nitrate or both), filipin, fluconazole, flucytosine, griseofulvin, haloprogin, hamycin, iodoquinol, isavuconazole, isoconazole, itraconazole, ketoconazole, luliconazole, miconazole, miltefosine, natamycin, nystatin, omoconazole, orotomide, oxiconazole, piroctone olamine, posaconazole, potassium iodide, propiconazole, ravuconazole, rimocidin, selenium disulfide, sertaconazole, sodium thiosulfate, sulconazole, sulfur, terconazole, tioconazole, tolnaftate, triacetin, undecylenic acid, voriconazole, or zinc pyrithione, or pharmaceutically acceptable salts thereof.

In some embodiments, the therapeutic agent is an antihelminth, for example, albendazole, albenza, biltricide, diethylcarbamazine, emverm, hetrazan, ivermectin, mebendazole, pin rid, pin x, praziquantel, pyrantel pamoate, stromectol, or vermox, or pharmaceutically acceptable salts thereof.

In some embodiments, the therapeutic agent is an antiviral, for example, abacavir, acyclovir (aciclovir), adefovir, amantadine, amprenavir(agenerase), ampligen, arbidol, atazanavir, atripla, balavir, cidofovir, combivir, dolutegravir, darunavir, delavirdine, didanosine, docosanol, edoxudine, efavirenz, emtricitabine, enfuvirtide, entecavir, ecoliever, famciclovir, fixed dose combination (antiretroviral), fomivirsen, fosamprenavir, foscarnet, fosfonet, fusion inhibitor, ibacitabine, imunovir, idoxuridine, imiquimod, indinavir, inosine, integrase inhibitor, interferon type iii, interferon type ii, interferon type i, interferon, lamivudine, lopinavir, loviride, maraviroc, moroxydine, methisazone, nelfinavir, nevirapine, nexavir, nitazoxanide, nucleoside analogues, norvir, oseltamivir, peginterferon alfa-2a, penciclovir, peramivir, pleconaril, podophyllotoxin, protease inhibitor, raltegravir, reverse transcriptase inhibitor, ribavirin, rimantadine, ritonavir, pyramidine, saquinavir, sofosbuvir, stavudine, synergistic enhancer (antiretroviral), telaprevir, tenofovir, tenofovir disoproxil, tipranavir, trifluridine, trizivir, tromantadine, truvada, valaciclovir (valtrex), valganciclovir, vicriviroc, vidarabine, viramidine, zalcitabine, zanamivir (relenza), or zidovudine, or a pharmaceutically acceptable salt thereof.

In some embodiments, the therapeutic agent is an NSAID. As used herein, the acronym “NSAID” refers to “non-steroidal anti-inflammatory.” In some embodiments, the NSAID is ibuprofen, aspirin, ketoprofen, sulindac, naproxen, etodolac, fenoprofen, diclofenac, flurbiprofen, ketorolac, piroxicam, indomethacin, mefenamic acid, meloxicam, nabumetone oxaprozin, ketoprofen, meclofenamate, tolmetin, or salsalate, or a pharmaceutically acceptable salt thereof.

Compositions of the invention may be designed to be administered to the skin or mucosal tissue of a patient in need of treatment. Compositions of the invention may be formulated as gels, transdermal patches, lotions, creams, sprays, mists, emulsions, or dispersions. Appropriate excipients for formulating a gel, transdermal patch, lotion, cream, spray, or mist are readily apparent to a person of skill in the art and include, but are not limited to, stabilizers, emulsifiers, thickeners, antimicrobials, humectants, propellants, spreading agents, polymers, and adhesives, such as pressure sensitive adhesives. In particular, excipients that may be used to form a transdermal gel include, but are not limited to, alcohols, glycols, glycerin, butylated hydroxytoluene (BHT), and water.

Also, within the scope of the disclosure are methods comprising applying any of the described compositions to the skin of a mammal for a time and under conditions effective to achieve passage of at least a portion of the composition into the skin. Skin permeation can be measured using techniques known in the art.

Without intending to be bound by theory, some embodiments of the claimed compositions are believed to kill microbiota on the surface of the skin or within the layers of the skin. Thus, the compositions of the disclosure can be used to administer an antiseptic (e.g., iodine), an antibiotic, an antifungal agent, an antihelminth agent, or an antiviral agent, to a mammal. For example, in preferred embodiments, these methods comprise applying any of the described compositions to the skin of a mammal for a time sufficient to achieve permeation of at least a portion of the antiseptic (e.g., iodine), an antibiotic, an antifungal agent, an antihelminth agent, or an antiviral agent into the skin. The extent to which a therapeutic agent permeates skin can be measured using techniques known in the art.

The present invention provides methods of killing microbiota, comprising contacting the microbiota with a described composition in an amount effective to kill the microbiota. As used here, microbiota refers to one or more of bacteria, archaea, protists, fungi and viruses.

The present invention also provides methods of treating viral or bacterial infections. In some embodiments, the present invention provides methods of treating a viral skin infection in a mammal, said methods comprising applying to the infected skin an amount of the described composition effective to decrease the viral load. As used herein, the term “viral load” refers to the quantity of virus per unit quantity of skin tissue. Methods of determining viral load in skin tissue are known to those of skill in the art. The methods of the invention decrease the viral load in the skin tissue, meaning that the viral load after applying the compositions of the invention is lower than the viral load prior to applying the compositions. In some embodiments, the viral load is decreased by 40%, 50%, 60%, 70%, 75%, 80%, 85%, 90%, 95%, 98%, or 99%, relative to the initial viral load prior to application of any composition of the disclosure. In other embodiments, the viral load is decreased such that the virus is undetectable.

In some embodiments, the viral skin infection is manifest by warts. In some embodiments, the present invention provides methods of reducing or eliminating warts from the skin of a mammal, said methods comprising applying to the warts an amount of the described composition effective to reduce or eliminate the warts. In some embodiments, the warts are common warts. In other embodiments, the warts are genital warts. In some embodiments, the warts are penile warts.

In other embodiments, the present invention provides methods of treating a bacterial skin infection in a mammal, said methods comprising applying to the infected skin an amount of the described composition effective to decrease the bacterial load. As used herein, the term “bacterial load” refers to the quantity of bacteria per unit quantity of skin tissue. Methods of determining bacterial load in skin tissue are known to those of skill in the art. The methods of the invention decrease the bacterial load in the skin tissue, meaning that the bacterial load after applying the compositions of the invention is lower than the bacterial load prior to applying the compositions. In some embodiments, the bacterial load is decreased by 40%, 50%, 60%, 70%, 75%, 80%, 85%, 90%, 95%, 98%, or 99%, relative to the initial bacterial load prior to application of any composition of the disclosure. In other embodiments, the bacterial load is decreased such that the bacteria is undetectable.

In some embodiments, the present invention provides method of reducing or eliminating MRSA infection from the skin of a mammal, said methods comprising applying to the infected skin an amount of the described composition effective to reduced or eliminate the MRSA. As used herein, MRSA refers to methicillin-resistant Staphylococcus aureus, a type of Staphylococcus bacteria that is resistant to many of the antibiotics used to treat ordinary Staphylococcus infections.

The compositions described herein can be applied to any accessible skin surface. Skin surfaces of interest include, but are not limited to: arms, leg, torso, head, neck, etc. The surface area that is covered by the transdermal formulation following application is generally sufficient to provide for the desired amount of agent administration, and in certain embodiments ranges from about 1 cm² to about 200 cm².

The compositions described herein can be applied a single time or a plurality of times over a given time period, e.g., the course of the disease condition being treated, where the dosing schedule when a plurality of patches are administered over a given time period may be daily, weekly, biweekly, monthly, etc. In some embodiments, the treatment is seven days.

The compositions of the disclosure will, in some embodiments, include, in addition to the above-discussed components, one or more additional components. Additional components include, but are not limited to, a transdermal absorption enhancer, a preservative (e.g., paraben), an antioxidant, a stabilizing agent, a filling agent that contains a hydrophilic polymer; a cross-linking agents; and a plasticizing agent.

The following examples are provided to illustrate the compositions, processes, and properties of the present disclosure. The examples are merely illustrative and not intended to limit the disclosure to the materials, conditions, or process parameters set forth therein.

### EXAMPLES

### Example 1. Iodine Composition

Nonaethylene glycol monododecyl ether (100%; 3 mL; 32 vol. %), 1-methyl-2-pyrrolidone (99.5%; 0.3 mL; 3 vol. %), ethanol (abs.; 4 mL; 43 vol. %), linoleic acid (67%; 1 mL; 7 vol. %); and iodine (1 mL; 7% in potassium iodide solution) are combined to form an admixture. The resulting composition is applied to an area of the skin that is infected with virus or bacteria.

### Example 2. Iodine Composition with an Antibiotic

Nonaethylene glycol monododecyl ether (100%; 3 mL; 32 vol. %), 1-methyl-2-pyrrolidone (99.5%; 0.3 mL; 3 vol. %), ethanol (abs.; 4 mL; 43 vol. %), linoleic acid (67%; 1 mL; 7 vol. %); iodine (1 mL; 7% in potassium iodide solution); and amoxicillin (20 mg/mL) are combined to form an admixture. The resulting composition is applied to an area of the skin that is infected with bacteria.

### Example 3. Iodine Composition

Nonaethylene glycol monododecyl ether (100%; 3 mL), 1-methyl-2-pyrrolidone (99.5%; 0.3 mL), ethanol (abs.; 4 mL), and linoleic acid (67%; 1 mL) are combined to form an admixture. One milliliter of the solution is diluted with 9 mL of ethanol or water. Iodine is added to give 1.5 vol. % in the mixture. The resulting composition is applied to an area of the skin that is infected with virus or bacteria.

### Example 4. Composition with an Antibiotic

Nonaethylene glycol monododecyl ether (100%; 3 mL), 1-methyl-2-pyrrolidone (99.5%; 0.3 mL), ethanol (abs.; 4 mL), and linoleic acid (67%; 1 mL) are combined to form an admixture. One milliliter of the mixture is mixed with 9 mL of ethanol or water. Amoxicillin (20 mg/mL) is added to the mixture. The resulting composition is applied to an area of the skin that is infected with bacteria.

### Example 5. Iodine Composition

Nonaethylene glycol monododecyl ether (100%; 3 mL), 1-methyl-2-pyrrolidone (99.5%; 0.3 mL), ethanol (abs.; 4 mL), and linoleic acid (67%; 1 mL) are combined to form an admixture. One milliliter of the solution is diluted with 99 mL of ethanol or water. Iodine is added to give 1.5 vol. % in the mixture. The resulting composition is applied to an area of the skin that is infected with virus or bacteria.

### Example 6. Composition with an Antibiotic

Nonaethylene glycol monododecyl ether (100%; 3 mL), 1-methyl-2-pyrrolidone (99.5%; 0.3 mL), ethanol (abs.; 4 mL), and linoleic acid (67%; 1 mL) are combined to form an admixture. One milliliter of the mixture is mixed with 99 mL of ethanol or water. Amoxicillin (20 mg/mL) is added to the mixture. The resulting composition is applied to an area of the skin that is infected with bacteria.

### Example 7. Treatment of Warts

An individual with common warts on eight of his ten fingers topically applies the composition of Example 1 to his warts daily for seven consecutive days. After seven days, the warts are significantly reduced or completely gone.

### Example 8. Treatment of MRSA

An individual with a MRSA infection on his skin topically applies the composition of Example 1 to the infected skin for seven consecutive days. After seven days, the MRSA infection is significantly reduced or completely gone.

What is claimed:

1. A composition comprising
a first component comprising:
a compound of formula I
R—(OCH₂CH₂)_{y}—OH (I)
wherein R is C₁₋₂₀alkyl, C₂₋₂₀alkenyl; or C₂₋₂₀alkynyl; and y is 1 to 25;
a tetrafunctional block copolymer surfactant terminating in primary hydroxyl groups;
a sorbitan derivative;
a C₈₋₁₀alkyl ammonium salt;
a compound of formula II
HO—(CH₂CH₂O)ₘ—C(CH₃)(C₄H₉)—C≡C—C(CH₃)(C₄H₉)—(OCH₂CH₂)ₙ—OH (II)
wherein m and n are each independently 1 to 25;
or a combination thereof;
a second component comprising:
an amide of the formula III
R²—N(R¹)—C(O)—R³ (III)
wherein
each R¹ is independently H or C₁₋₃alkyl; and
R² and R³ are independently C₁₋₇alkyl or together with the atoms to which they are attached, form a lactam having 3 to 10 carbon atoms;
a sulfoxide;
a urea;
ethyl acetate;
or a combination thereof;
a C₁₋₁₀ alkyl alcohol;
an organic acid having 1 to 25 carbon atoms;
optionally, water; and
a therapeutic agent which is an antiseptic, an antibiotic, an antifungal agent, an antihelminth agent, an antiviral agent, or an NSAID.

2. The composition of claim 1, wherein the therapeutic agent is an antiseptic.

3. The composition of claim 2, wherein the antiseptic is iodine, chlorhexidine gluconate, octenidine dihydrochloride, polyhexamethylene biguanide, or boric acid, or a pharmaceutically acceptable salt thereof.

4. The composition of claim 3, wherein the antiseptic is iodine.

5. The composition of claim 1, wherein the therapeutic agent is an antibiotic.

6. The composition of claim 5, wherein the antibiotic is amikacin, aminoglycosides, amoxicillin, amoxicillin/clavulanate, ampicillin, ampicillin/sulbactam, arsphenamine, azithromycin, azlocillin, aztreonam, bacitracin, bactrium, capreomycin, carbapenems, cefaclor, cefadroxil, cefalexin, cefamandole, cefazolin, cefdinir, cefditoren, cefepime, cefepime, cefixime, cefmetazole, cefonicid, cefoperazone, cefotaxime, cefotetan, cefoxitin, cefpodoxime, cefprozil, ceftaroline, ceftaroline fosamil, ceftazidime, ceftazidime, ceftibuten, ceftizoxime, ceftobiprole, ceftobiprole, ceftobiprole, ceftolozane/tazobactam, ceftriaxone, cefuroxime, cephalothin, cephapirin, cephradine, chloramphenicol, ciprofloxacin, clarithromycin, clindamycin, clindamycin, clofazimine, colistin, cycloserine, dalbavancin, dalbavancin, dapsone, daptomycin, daptomycin, daptomycin, demeclocycline, dicloxacillin, doripenem, doxycycline, doxycyline, enoxacin, ertapenem, erythromycin, ethambutol, ethionamide, fidaxomicin, flucloxacillin, fluoroquinolones, fosfomycin, furazolidone, fusidic acid, fusidic acid, gatifloxacin, geldanamycin, gemifloxacin, gentamicin, grepafloxacin, herbimycin, imipenem/cilastatin, isoniazid, kanamycin, levofloxacin, lincomycin, linezolid, linezolid, linezolid, lomefloxacin, loracarbef, loracarbef, mafenide, meropenem, metacycline, methicillin, metronidazole, mezlocillin, minocycline, moxalactam, moxifloxacin, mupirocin, mupirocin, nadifloxacin, nafcillin, nalidixic acid, neomycin, netilmicin, nitrofurantoin, norfloxacin, ofloxacin, oritavancin, oritavancin, oxacillin, oxytetracycline, paromomycin, penicillin g, penicillin g, penicillin v, piperacillin, piperacillin/tazobactam, piperacillin/tazobactam, platensimycin, polymyxin b, posizolid, pyrazinamide, quinupristin/dalfopristin, radezolid, rifabutin, rifampicin, rifapentine, rifaximin, roxithromycin, silver sulfadiazine, sparfloxacin, spectinomycin, spiramycin, streptogramins, streptomycin, streptomycin, sulfacetamide, sulfadiazine, sulfadimethoxine, sulfamethizole, sulfamethoxazole, sulfanilimide, sulfasalazine, sulfisoxazole, sulfonamidochrysoidine, tedizolid, teicoplanin, telavancin, telavancin, telithromycin, temafloxacin, temocillin, tetracycline, thiamphenicol, ticarcillin, ticarcillin/clavulanate, ticarcillin/clavulanic acid, tigecycline, tigecycline, tigecycline, tinidazole, tobramycin, torezolid, trimethoprim, trimethoprim-sulfamethoxazole (co-trimoxazole) (tmp-smx), trovafloxacin, vancomycin, or a pharmaceutically acceptable salt thereof.

7. The composition of claim 5, wherein the antibiotic is vancomycin, bactrium, doxycyline, ceftobiprole, ceftaroline, clindamycin, dalbavancin, daptomycin, fusidic acid, linezolid, mupirocin, oritavancin, tedizolid, telavancin, or tigecycline, or a pharmaceutically acceptable salt thereof.

8. The composition of claim 1, wherein the therapeutic agent is an antifungal agent.

9. The composition of claim 8, wherein the antifungal agent is abafungin, acrisorcin, albaconazole, amorolfin, butenafine, naftifine, terbinafine, amphotericin b, anidulafungin, aurones, benzoic acid, bifonazole, butoconazole, candicidin, caspofungin, castellani's paint, ciclopirox, clioquinol, clotrimazole, coal tar, copper(ii) sulfate^{II}, crystal violet, econazole, efinaconazole, epoxiconazole, fenticonazole (base, nitrate or both), filipin, fluconazole, flucytosine, griseofulvin, haloprogin, hamycin, iodoquinol, isavuconazole, isoconazole, itraconazole, ketoconazole, luliconazole, miconazole, miltefosine, natamycin, nystatin, omoconazole, orotomide, oxiconazole, piroctone olamine, posaconazole, potassium iodide, propiconazole, ravuconazole, rimocidin, selenium disulfide, sertaconazole, sodium thiosulfate, sulconazole, sulfur, terconazole, tioconazole, tolnaftate, triacetin, undecylenic acid, voriconazole, or zinc pyrithione, or a pharmaceutically acceptable salt thereof.

10. The composition of claim 1, wherein the therapeutic agent is an antihelminth agent.

11. The composition of claim 10, wherein the antihelminth agent is albendazole, albenza, biltricide, diethylcarbamazine, emverm, hetrazan, ivermectin, mebendazole, pin rid, pin x, praziquantel, pyrantel pamoate, stromectol, or vermox, or a pharmaceutically acceptable salt thereof.

12. The composition of claim 1, wherein the therapeutic agent is an antiviral agent.

13. The composition of claim 12, wherein the antiviral agent is abacavir, acyclovir (aciclovir), adefovir, amantadine, amprenavir (agenerase), ampligen, arbidol, atazanavir, atripla, balavir, cidofovir, combivir, dolutegravir, darunavir, delavirdine, didanosine, docosanol, edoxudine, efavirenz, emtricitabine, enfuvirtide, entecavir, ecoliever, famciclovir, fixed dose combination (antiretroviral), fomivirsen, fosamprenavir, foscarnet, fosfonet, fusion inhibitor, ibacitabine, imunovir, idoxuridine, imiquimod, indinavir, inosine, integrase inhibitor, interferon type iii, interferon type ii, interferon type i, interferon, lamivudine, lopinavir, loviride, maraviroc, moroxydine, methisazone, nelfinavir, nevirapine, nexavir, nitazoxanide, nucleoside analogues, norvir, oseltamivir, peginterferon alfa-2a, penciclovir, peramivir, pleconaril, podophyllotoxin, protease inhibitor, raltegravir, reverse transcriptase inhibitor, ribavirin, rimantadine, ritonavir, pyramidine, saquinavir, sofosbuvir, stavudine, synergistic enhancer (antiretroviral), telaprevir, tenofovir, tenofovir disoproxil, tipranavir, trifluridine, trizivir, tromantadine, truvada, valaciclovir (valtrex), valganciclovir, vicriviroc, vidarabine, viramidine, zalcitabine, zanamivir (relenza), or zidovudine, or a pharmaceutically acceptable salt thereof.

14. The composition of claim 1, wherein the therapeutic agent is an NSAID.

15. The composition of claim 14, wherein the NSAID is ibuprofen, aspirin, ketoprofen, sulindac, naproxen, etodolac, fenoprofen, diclofenac, flurbiprofen, ketorolac, piroxicam, indomethacin, mefenamic acid, meloxicam, nabumetone oxaprozin, ketoprofen, meclofenamate, tolmetin, or salsalate, or a pharmaceutically acceptable salt thereof.

16. The composition of claim 1, wherein said first component is cetomacrogol 1000; octadecan-1-ol, ethoxylated; polyoxyethylene(12)tridecyl ether; polyoxyethylene(10)tridecyl ether; fatty alcohol polyoxyethylene ether, polyoxyethylene branched nonylcyclohexyl ether, nonaethylene glycol monododecyl ether, 23-{[4-(2,4,4-trimethyl-2-pentanyl)cyclohexyl]oxy}-3,6,9,12,15,18,21-heptaoxatricosan-1-ol, or a combination thereof.

17. The composition of claim 16, wherein said first component is nonaethylene glycol monododecyl ether.

18. The composition of claim 1, wherein the second component is an amide of formula III.

19. The composition of claim 18, wherein R² and R³, together with the atoms to which they are attached, form a lactam having 3 to 10 carbon atoms.

20. The composition of claim 19, wherein the lactam is a pyrrolidone.

21. The composition of claim 20, wherein the pyrrolidone is 1-methyl-2-pyrrolidinone.

22. The composition of claim 1, wherein the C₁₋₁₀alkyl alcohol is methanol, glycerol, propylene glycol, ethanol, isopropanol, 1-propanol, butanol, t-butanol, pentanol, 1-octanol, or a combination thereof.

23. The composition of claim 1, wherein the organic acid is a fatty acid or a C₁₋₆alkyl acid.

24. The composition of claim 23, wherein the fatty acid is linoleic acid.

25. The composition of claim 1, wherein the first component is nonaethylene glycol monododecyl ether; the second component is 1-methyl-2-pyrrolidinone; and the organic acid is linoleic acid.

26. The composition of claim 1 comprising
about 32-36 vol. % of the first component;
about 2-4 vol. % of the second component;
about 40-48 vol. % of the C₁₋₁₀alkyl alcohol;
about 6-12 vol. % of the organic acid; and
a therapeutic agent which is an antiseptic, an antibiotic, an antifungal agent, an antihelminth agent, an antiviral agent, or an NSAID.

27. The composition of claim 1 comprising
about 32 vol. % of nonaethylene glycol monododecyl ether;
about 3 vol. % of 1-methyl-2-pyrrolidone;
about 43 vol. % of ethanol;
about 7 vol. % of linoleic acid; and
a therapeutic agent which is an antiseptic, an antibiotic, an antifungal agent, an antihelminth agent, an antiviral agent, or an NSAID.

28. The composition of claim 1 comprising
about 3.2-3.6 vol. % of the first component;
about 0.2-0.4 vol. % of the second component;
about 4.0-4.8 vol. % of the C₁₋₁₀alkyl alcohol;
about 0.6-1.2 vol. % of the organic acid; 85-91 vol. % ethanol or water, and
a therapeutic agent which is an antiseptic, an antibiotic, an antifungal agent, an antihelminth agent, an antiviral agent, or an NSAID.

29. The composition of claim 1 comprising
about 0.32-0.36 vol. % of the first component;
about 0.02-0.04 vol. % of the second component;
about 0.40-0.48 vol. % of the C₁₋₁₀alkyl alcohol;
about 0.06-0.12 vol. % of the organic acid; 90-99 vol. % ethanol or water, and
a therapeutic agent which is an antiseptic, an antibiotic, an antifungal agent, an antihelminth agent, an antiviral agent, or an NSAID.

30. The composition of claim 1 in the form of a gel, transdermal patch, lotion, cream, spray, emulsion, or dispersion.

31. A method comprising applying a composition of claim 1 to the skin of a mammal for a time sufficient to achieve permeation of at least a portion of the iodine into the skin.

32. A method comprising applying a composition of claim 1 to the skin of a mammal for a time and under conditions effective to achieve passage of at least a portion of said composition into said skin.

33. A method of killing microbiota comprising contacting the microbiota with an amount of a composition of claim 1 effective to kill said microbiota.

34. A method of treating viral skin infection in a mammal comprising applying to the virus-infected skin of said mammal an amount of the composition of claim 1 effective to decrease the viral load.

35. The method of claim 34, wherein said viral skin infection is manifested by warts.

36. A method of treating a bacterial skin infection in a mammal comprising applying to the bacteria-infected skin of said mammal an amount of the composition of claim 1 effective to decrease the bacterial load.

37. The method of claim 34, wherein said bacterial skin infection is a MRSA infection.

38. An iodophore comprising iodine and N-methyl-2-pyrrolidone.

39. The composition of claim 1, further comprising an iodophore comprising iodine and N-methy-2-pyrrolidone.

40. The composition of claim 39, further comprising a PVP-iodine iodophore.

41. The composition of claim 1, comprising a therapeutic agent that is an antiseptic, and a therapeutic agent that is an antibiotic.

42. The composition of claim 41, wherein the antiseptic is iodine and the antibiotic is vancomycin.

43. The composition of claim 41 wherein the antiseptic is iodine and the antibiotic is amoxicillin.
